(19) United States
(12) Patent Application Publication
(10) Pub. No.: US 2005/0245932 A1
(43) Pub. Date: Nov. 3, 2005

(54) Apparatus and methods for securing tissue to bone

(76) Inventors: Gary S. Fanton, Portola Valley, CA (US); John E. Ashley, San Francisco, CA (US)

Correspondence Address: HOEKENDIJK & LYNCH, LLP, P.O. BOX 4787, BURLINGAME, CA, 94011-4787, US

(21) Appl. No.: 11/107,372

(22) Filed: Apr. 15, 2005

Related U.S. Application Data
(60) Provisional application No. 60/562,778, filed on Apr. 16, 2004.

(51) Int. Cl. A61B 17/58

(52) U.S. Cl. 606/72

(57) ABSTRACT

The present invention relates to apparatus and methods for securing tissue to bone using a suture anchoring system that provides enhanced tactile feedback and does not require tying a suture knot. In each embodiment, a surgeon can individually tension the free ends of the suture to fine-tune the placement of the tissue with respect to the bone, and then secure the suture without tying a knot. In several embodiments of the present invention, the device may be transformed between locked and unlocked suture states, thereby allowing further fine-tuning of the tension in the suture.

### FIELD OF THE INVENTION

The present invention relates to the field of surgical arthroscopy, and more particularly, to apparatus and methods for facilitating the attachment of tissue to bone using a suture anchoring system that provides enhanced tactile feedback and does not require tying a suture knot.

### BACKGROUND OF THE INVENTION

Many attempts have been made to provide devices that allow the arthroscopic securing of torn tissue to a substrate bone. For example, there have been numerous devices designed for the shoulder to allow a torn rotator cuff to be secured to the humeral head.

Typically, in a first step, a hole is drilled into the bone under arthroscopic visualization. A length of a suture generally is employed to permit securing of the tissue to the bone. The suture length is threaded through a portion of the tissue, and also is coupled to a bone anchor configured to be inserted into the hole in the bone. One or both of the suture ends may extend outside of the arthroscopic site, so that the suture can be manipulated by a physician.

Once the suture is coupled between the tissue and the bone anchor, the bone anchor is inserted into the hole. The bone anchor generally is configured to lock itself within the hole in the bone upon deployment therein. Several means for securing the bone anchor within the hole of a bone are known in the art.

Once the bone anchor is secured within the hole in the bone, a physician may tension one or both ends of the suture to approximate the positioning of the tissue with respect to the bone. Once the tissue is positioned as desired, the suture is locked in place to maintain the tension in the suture. The free end or ends of the suture then are clipped under arthroscopic visualization to complete the procedure.

There are various drawbacks associated with such previously-known suture anchoring systems. For example, many of the previously-known systems require the physician to tie a knot to lock the suture, thereby maintaining the tensile forces that hold the tissue in place. However, when performing the procedure under arthroscopic visualization and having minimal clearance, it is often difficult for the surgeon to perform the maneuvers necessary to tie a knot in the confined working space.

Further, previously-known suture anchoring systems generally do not allow the surgeon direct tactile feedback of the tension in the suture between the tissue and the bone. For example, in those systems where only one free end of the suture may be manipulated by a physician, difficulties may arise in approximating the position of the tissue with respect to the bone. By contrast, when a physician can manipulate both ends of a suture independently, two different forces may be applied to the tissue to facilitate positioning of the tissue with respect to the bone.

Still other previously-known suture anchoring systems have relied on urging tissue towards a bone anchor by tightening a knot. In such systems, the suture is threaded through tissue and a knot is tied proximal to the tissue. As the knot is tightened, the tissue is pushed towards the bone. However, such systems have various drawbacks, including not being able to manually determine the tension of the tissue, and also risking the possibility that the knot will become embedded within the tissue.

An example of a previously-known method and apparatus for attaching tissue to bone using a knotless suture anchoring device is described in U.S. Pat. No. 6,585,730 to Foerster. Foerster describes a device having a distal anchor portion and a wedge body. A suture length has a bound end and a free end. The bound end of the suture is coupled to the tissue, and the suture extends around the wedge body at the distal end of the device, such that a free end of the suture may be manipulated by a physician.

Once the distal anchor portion is secured within the bone, the practitioner pulls the free end of the suture to draw the soft tissue towards the bone. Tension in the suture draws the wedge body up into the lumen of the distal anchor portion. At this time, the length of suture wrapped around the wedge body becomes pinched between the wedge body and the distal anchor portion.

The Foerster patent suggests that the pinching force imposed upon the suture creates a self-locking mechanism. Further, the patent suggests that applying a tensile force to the free suture end, after it has been clamped, will cause the wedge body to move distally to unlock the previously-pinched suture and enable “reversibility” of the device for further fine-tuning.

The device described in the Foerster patent has several drawbacks. First, the device appears to rely on tension alone to secure the tissue to the bone. Specifically, merely tensioning the free end of the suture is expected to lock the device, since the suture is clamped between the wedge body and the distal anchor portion. Then, the Foerster patent suggests that simply pulling the free end of the suture will unlock the device, since the pinched suture wants to straighten out when the free end is tensioned. Therefore, the device is both locked and unlocked by tensioning the free end of the suture. Accordingly, it is possible that incidental tensile forces applied to the free suture end may unexpectedly unlock the device. In short, when tensioning the free end of the suture is the means for locking and unlocking the device, it may be difficult to lock the device in a desired position, or the device may come unlocked at an undesirable time.

Another drawback of the device described in the Foerster patent is that one of the suture ends is “bound” to the tissue. It is expected that if a physician can tension both ends of the suture, it will facilitate positioning of the tissue with respect to the bone.

Another previously-known knotless suture anchor is described in U.S. Pat. No. 6,692,516 to West et al. (“West”). The embodiment of FIGS. 11-17 of the West patent describes a device having a shaft with a distal crown portion. The shaft is disposed through an outer member, such that the crown portion is disposed distal to the outer member.

The shaft has an elongated opening through which the two free suture ends can be threaded. Therefore, in use, the suture is threaded through tissue to form a loop, and the two free ends of the suture are threaded through the elongated opening in the shaft, such that the free ends then can be manipulated by a physician.

In operation, a physician approximates the positioning of the tissue with respect to the bone. A proximally-directed force then is applied to the shaft to cause the shaft to move proximally with respect to the outer member. This causes the suture, which is threaded through the opening in the shaft, to be pinched between the shaft and the outer member, thereby locking the suture in place. At the same time, the proximal retraction of the shaft with respect to the outer member causes radially expandable fingers on the crown portion to be deployed outward, thereby securing the device within the hole in the bone.

The device described in the West patent does not appear to permit suture adjustments after the suture is locked in place. This is because the proximal retraction of the shaft with respect to the outer member both pinches the suture in place, and also deploys the expandable fingers to secure the device in the borehole. Therefore, it is not possible to adjust the suture further because it would be necessary to distally advance the shaft to do so, i.e., to remove the compressive force imposed upon the suture. However, the shaft cannot be advanced distally because the expandable members, secured within the bone, would prohibit such movement.

In view of these drawbacks of previously known suture anchoring systems, it would be desirable to provide apparatus and methods for securing tissue to bone that are easy to use and do not require a large incision.

It further would be desirable to provide apparatus and methods for securing tissue to bone that allow a surgeon direct tactile feedback of the tension in the suture between the tissue and the bone.

It also would be desirable to provide apparatus and methods for securing tissue to bone that allow a surgeon to tension both ends of a suture individually to fine-tune the placement of the tissue with respect to the bone.

It still further would be desirable to provide apparatus and methods for securing tissue to bone that allows a suture to be locked in place without tying a knot.

### SUMMARY OF THE INVENTION

In view of the foregoing, it is an object of the present invention to provide apparatus and methods for securing tissue to bone that are easy to use and do not require a large incision.

It is also an object of the present invention to provide apparatus and methods for securing tissue to bone that allow a surgeon direct tactile feedback of the tension in the suture between the tissue and the bone.

It is a further object of the present invention to provide apparatus and methods for securing tissue to bone that allow a surgeon to tension both ends of a suture individually to fine-tune the placement of the tissue with respect to the bone.

It is still a further object of the present invention to provide apparatus and methods for securing tissue to bone that allow a suture to be locked in place without tying a knot.

These and other objects of the present invention are accomplished by providing apparatus comprising a bone anchor member configured to be securely disposed in a hole drilled in a bone. A suture length may be coupled between the bone anchor member and tissue, or alternatively, between a plug portion that fits within a bore of the bone anchor member and the tissue. In each embodiment, a surgeon can individually tension each end of the suture to fine-tune the placement of the tissue with respect to the bone, and then secure the suture without tying a knot.

In a first embodiment of the present invention, the apparatus comprises a bone anchor member comprising first and second passages that extend laterally through the bone anchor member. A suture is threaded through the first passage, then threaded through the tissue, and finally threaded back through the second passage, such that the first and second free ends of the suture can be manipulated by a physician. Alternatively, the suture can be threaded through the tissue first, such that the free ends extend from the tissue. The free ends then are threaded through the respective first and second passages of the bone anchor member.

In this embodiment, the first and second passages each comprise a plurality of cleated members that are configured to permit one-way movement of the first and second suture ends, i.e., each suture end can be tensioned in a proximal direction. Accordingly, a physician can incrementally fine-tune the positioning of the tissue with respect to the bone by individually tensioning the suture ends. When a desired tension is achieved, as determined by tactile feedback, the suture ends are locked in place via the one-way cleated members.

In an alternative embodiment of the present invention, the bone anchor member comprises a bore disposed therein. The bore is configured to receive a plug portion, which may have various configurations. In one embodiment, the plug portion may comprises first and second passages having a plurality of cleated members. The plurality of cleated members are configured to permit one-way movement of the first and second suture ends in their respective passages.

The plug portion may be secured within the bone anchor bore using any number of means, as described hereinbelow. Either before or after the plug portion is secured within the bone anchor member, a physician may individually tension the first and second suture ends, which are disposed through the first and second one-way passages of the plug portion, to secure the tissue to the bone.

In further alternative embodiments of the present invention, the suture may be transformed between locked and unlocked state, as desired. In one of these fully reversible embodiments, the bone anchor member and the plug portion each comprise first and second laterally extending passages. When the plug portion is disposed within the bore of the bone anchor member, the first passage of the plug portion can align with the first passage of the bone anchor member, and the second passage of the plug portion can align with the second passage of the bone anchor member.

In this embodiment, a first suture end is threaded through the first passage of the plug portion and the first passage of the bone anchor member, while the second suture end is threaded through the second passage of the plug portion and the second passage of the bone anchor member. When the first and second passages of the plug portion are aligned with the first and second passages of the bone anchor member, respectively, then the first and second suture ends may be individually tensioned by a physician. When the passages of the plug portion and bone anchor member are misaligned, then the suture ends are pinched and locked in place.

In further alternative embodiments, the plug portion may be rotated with respect to the bone anchor member. When the plug portion is rotated in a first direction, the plug portion pinches the suture to lock the suture in place. When the plug portion is rotated in an opposing direction, the suture ends are unlocked and may be manipulated by a physician.

In still further alternative embodiments, the bone anchor member may comprise a flexible member disposed therein, and a laterally extending passage disposed distal to the flexible member. The first and second suture ends are configured to be threaded through the passage. When a physician desires to lock the suture in place, a distally-directed force is applied to the flexible member, via the bore, to cause the flexible member to pinch the suture ends and lock the suture in place. If a physician wishes to further adjust the suture ends, then the distally-applied force is removed, thereby allowing movement of the suture.

Alternatively, a threaded cap may be disposed within the bore of the bone anchor member, and configured for movement within a grooved inner portion of the bore. A passage through which the first and second suture ends passes is situated distal to the threaded cap. If a physician wishes to lock the suture in place, then the threaded cap is advanced distally within the bore, e.g., by rotating the cap in a first direction, to cause the cap to pinch the suture. If a physician wishes to unlock the suture, then the threaded cap is rotated in an opposing direction so that it is retracted proximally within the bore.

Several further embodiments of the present invention also are disclosed in detail hereinbelow. Each embodiment permits the incremental tensioning of first and second suture ends and locking of the suture without tying a knot.

Methods for using the apparatus of the present invention to facilitate the attachment of tissue to bone also are disclosed.

### BRIEF DESCRIPTION OF THE DRAWINGS

Further features of the invention, its nature and various advantages will be more apparent from the accompanying drawings and the following detailed description of the preferred embodiments, in which:

FIG. 1 is a schematic of a bone and tissue interface;

FIG. 2A-2C are, respectively, a side view, a front view, and a side-sectional view along line A-A of FIG. 2B showing a first embodiment of the present invention;

FIGS. 3A-3C are, respectively, a side-sectional view of an alternative embodiment of the present invention having a bone anchor member and a plug portion, a side view of the plug portion of FIG. 3A, and an opposing side view of the plug portion;

FIGS. 4A-4C are, respectively, a side-sectional view of a further alternative embodiment of the present invention having a bone anchor member and a plug portion, a side view of the plug portion of FIG. 4A, and an opposing side view of the plug portion;

FIG. 5 is a side sectional view of an alternative embodiment of the present invention having at least one adhesive delivery channel;

FIGS. 6A-6B are, respectively, a side-sectional view of a further alternative embodiment of the present invention, and the device of FIG. 6A shown deployed in a hole of a bone;

FIG. 7 is a side sectional view of a further alternative embodiment of the present invention;

FIG. 8 is a side view of an alternative embodiment of the present invention, which is configured for use with a through hole drilled in a bone;

FIGS. 9A-9C are side sectional views illustrating the use of an alternative embodiment of the present invention;

FIG. 10 is an alternative embodiment of the bone anchor member described in FIGS. 9A-9C;

FIGS. 11A-11C are, respectively, a top view of an alternative embodiment of the present invention in an unlocked state, a side sectional view of the device along line B-B of FIG. 11A, and a top view of the device of FIG. 11A in a locked state;

FIG. 12 illustrates use of a suture in connection with the embodiment described in FIGS. 11A-11C;

FIGS. 13A-13B are side sectional views of an alternative embodiment of the present invention in unlocked and locked states, respectively;

FIGS. 14A-14B are side sectional views of a further alternative embodiment of the present invention in unlocked and locked states, respectively;

FIGS. 15A-15B are side sectional views of yet a further alternative embodiment of the present invention in unlocked and locked states, respectively;

FIGS. 16A-16B are, respectively, a top sectional view and a side view of an alternative embodiment of the present invention in an unlocked state;

FIGS. 17A-17B are, respectively, a top sectional view and a side view of the embodiment of FIGS. 16A-16B in a locked state;

FIGS. 18A-18B are side sectional views of a further alternative embodiment of the present invention; and

FIGS. 19A-19C are, respectively, a side sectional view, a side view, and a bottom view of an alternative embodiment of the plug portion of FIGS. 18A-18B.

### DETAILED DESCRIPTION OF THE INVENTION

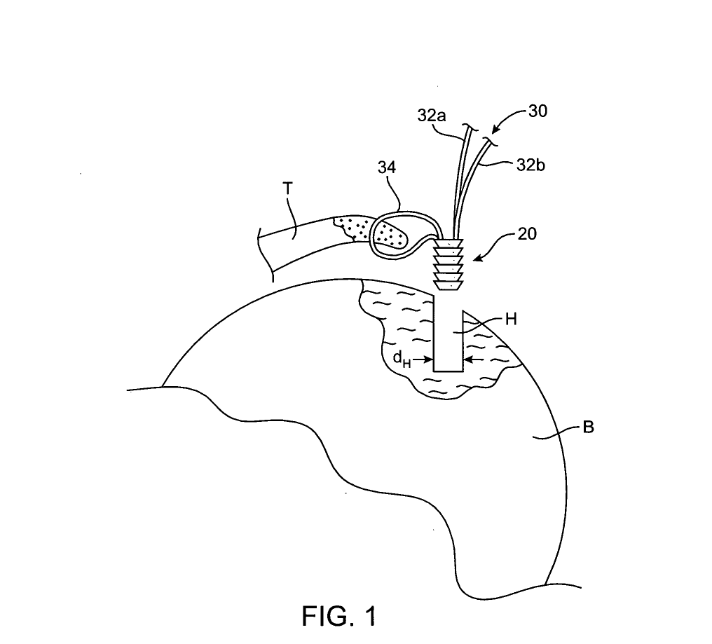

Referring now to FIG. 1, a schematic of a bone and tissue interface is shown primarily for illustrative purposes. In FIG. 1, tissue T has a torn end and it is desirable to secure the torn end to a section of bone B. In a first step, hole H having diameter d_{H} is drilled in bone B, as depicted, using techniques that are well known in the art.

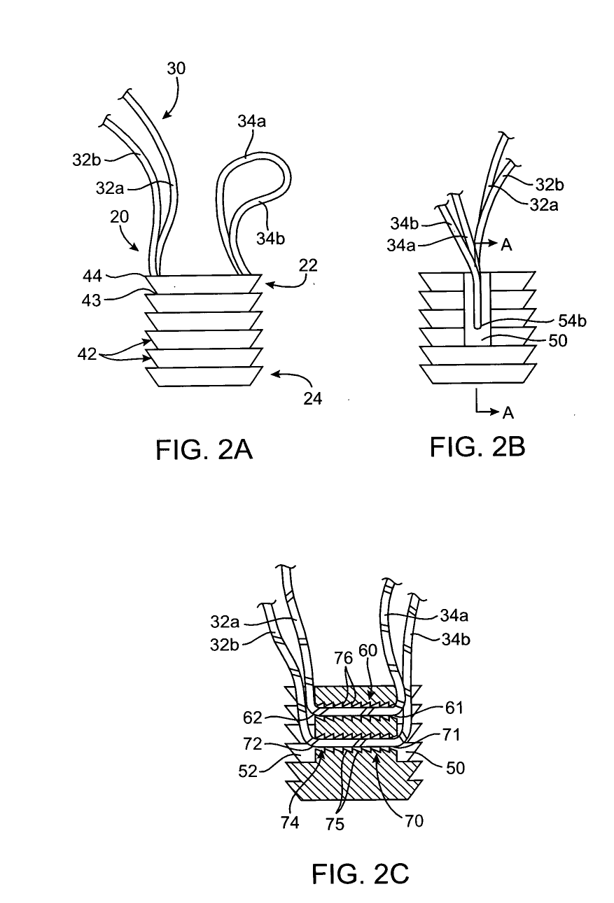

Bone anchor member 20, which will be described in greater detail in FIGS. 2A-2C hereinbelow, is shown as a means for securing tissue T to bone B. Bone anchor member 20 is configured to be used in conjunction with a suture length 30. Suture 30 has first and second ends 32a and 32b, which are coupled to bone anchor member 20, as described in FIGS. 2A-2C hereinbelow. It should be noted that a central region of suture 30 forms loop 34, which is threaded through a section of tissue T near the torn end of the tissue, as depicted in FIG. 1, using techniques that are known in the art.

Referring now to FIGS. 2A-2C, features of bone anchor member 20 are described in greater detail. Bone anchor member 20 has proximal region 22 and distal region 24, as depicted in FIG. 2A. The apparatus further comprises means for securing bone anchor member 20 within hole H of bone B (see FIG. 1). As depicted, the means for securing bone anchor member 20 comprises plurality of cleated members 42, which preferably are formed on or attached to an exterior surface of bone anchor member 20. Alternatively, other means for securing bone anchor member 20 within hole H may be used, such as radially expandable members (not shown) that dig into surrounding bone B, or threaded exterior members that screw into surrounding bone.

Referring now to FIGS. 2B-2C, bone anchor member 20 preferably further comprises first and second guide channels 50 and 52, respectively, which preferably are formed within opposing surfaces of bone anchor member 20. First and second guide channels 50 and 52 are configured to accommodate regions of suture 30, so that the suture regions do not extend outside of the confines of the guide channels when in use.

Bone anchor member 20 further comprises first and second passages 60 and 70. First and second passages 60 and 70 extend laterally through a main body of bone anchor member 20, as depicted in FIG. 2C. First passage 60 communicates with first guide channel 50 via opening 61, and further communicates with second guide channel 52 via opening 62. Similarly, passage 70 communicates with first guide channel 50 via opening 71, and further communicates with second guide channel 52 via opening 72.

In the embodiment of FIGS. 2A-2C, first passage 60 is shown disposed proximal to second passage 70, i.e., the first passage is closer to proximal region 22 of bone anchor member 20. However, as will be apparent to one skilled in the art, the passages also may be disposed adjacent one another, or otherwise positioned, to achieve the objects of the present invention.

First passage 60 and second passage 70 each comprise at least one cleated member 74. Each cleated member comprises angled sections 75 and substantially orthogonal sections 76, which are disposed adjacent one another, thereby forming a cleated shape, as shown in FIG. 2C.

The cleated members are configured such that angled sections 75 are angled towards openings 62 and 72 of passages 60 and 70, respectively, as shown in FIG. 2C. In accordance with one aspect of the present invention, cleated passages 60 and 70 are configured to permit oneway movement of first and second suture ends 32a and 32b, respectively. For example, when first end 32a is pulled in a proximal direction by a physician, angled sections 75 permit movement of the suture end in the proximal direction. However, a physician cannot distally advance suture end 32a within passage 60.

In a preferred embodiment, suture 30 has an outer diameter that is slightly larger than an inner diameter of cleated passages 60 and 70. Therefore, first and second suture ends 32a and 32b can pass through cleated passages 60 and 70 in a proximal direction with relatively little resistance. However, the suture will hold significantly greater force in the distal direction.

In a preferred method, a central region of suture 30 can be looped through tissue T first, such that free ends 32a and 32b extend from the tissue. Free end 32a then is threaded through one-way cleated passage 60 in a proximal direction, while free end 32b is threaded through one-way cleated passage 70, also in a proximal direction.

As will be apparent to one skilled in the art, suture 30 may be coupled between tissue T and bone anchor member 20 using other threading techniques, so long as the suture ultimately is situated in a manner depicted in FIG. 2C.

At this time, first end 32a of suture 30 is disposed through first passage 60, then transitions into loop portion 34a. Loop portion 34a transitions into loop portion 34b, forming loop 34 therebetween, which is coupled to tissue T (see FIG. 1). Loop portion 34b transitions into second end 32b, which is disposed through passage 70, as shown in FIG. 2C. Accordingly, first and second ends 32a and 32b of suture 30 may be independently manipulated by a physician for purposes described hereinbelow.

In operation, after suture 30 is coupled to bone anchor member 20 and tissue T as described hereinabove, bone anchor member 20 is distally advanced into hole H of bone B under arthroscopic guidance. Cleated members 42 of bone anchor member 20 allow the bone anchor member to be advanced distally within hole H when an appropriate force is applied, but cleated members 42 inhibit proximal movement of bone anchor member 20 to provide a secure anchor within hole H.

At this time, the surgeon can approximate the positioning of tissue T with respect to bone B (see FIG. 1) by pulling first end 32a and/or second end 32b proximally through cleated passages 60 and 70. Advantageously, the use of two separate passages allows the surgeon to tension each end of the suture independently, which is often desirable when tissue is torn irregularly.

Further, the use of a plurality of cleated passages 60 and 70 permits incremental tensioning of first and second suture ends 32a and 32b. This allows a physician to incrementally adjust the positioning of the tissue, using tactile feedback as a guide. Once a desired tension is achieved, the physician simply needs to stop retracting the suture ends, and the suture is automatically locked in place. Advantageously, there is no need to tie a knot.

In accordance with another object of the present invention, guide channels 50 and 52 permit the retraction of first and second suture ends 32a and 32b when bone anchor member 20 is secured within hole H by providing a clearance between the bone anchor member and the bone itself.

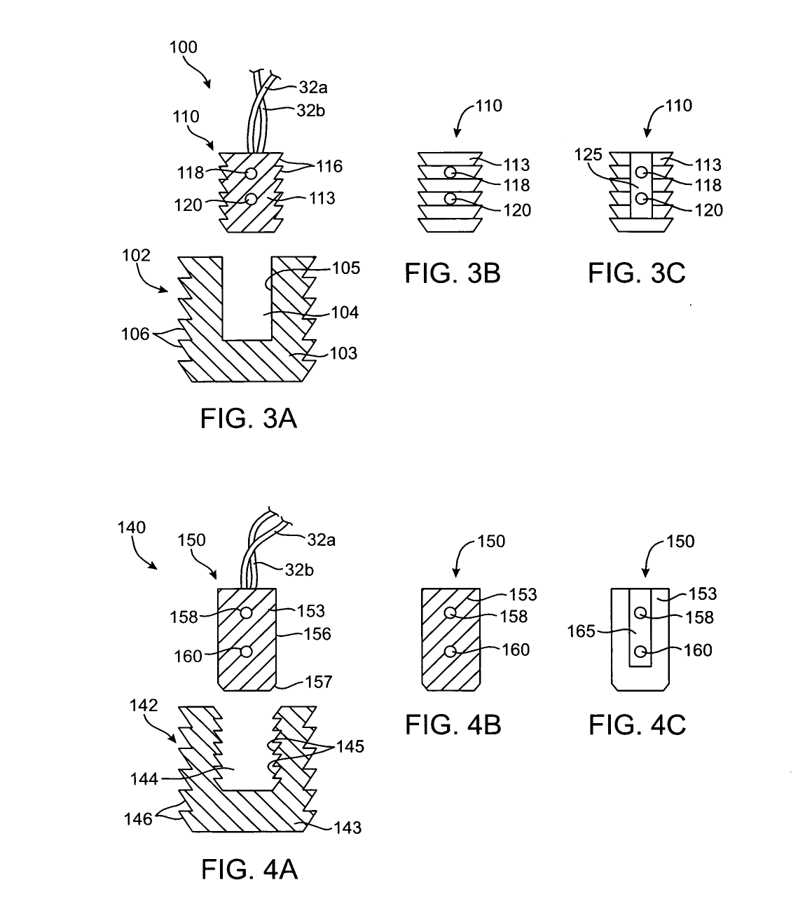

Referring now to FIGS. 3A-3C, an alternative embodiment of the present invention is described. In FIG. 3A, apparatus 100 comprises bone anchor member 102 and plug portion 110. Bone anchor member 102 comprises main body 103 having bore 104 disposed therein, as depicted in FIG. 3A. Further, main body 103 of bone anchor member 102 comprises exterior cleated members 106, which are similar to cleated members 42 of bone anchor member 20. Cleated members 106 are configured to be inserted into hole H of bone B (see FIG. 1) using a frictional force fit.

In operation, once bone anchor member 102 is secured in hole H, then plug portion 110 may be inserted into bore 104 of bone anchor member 102. Cleated members 116 of plug portion 110 are configured to permit distal advancement of the plug portion into bore 104, with some friction provided between cleated members 116 and inner wall 105. However, cleated members 116 ensure that plug portion 110 cannot be retracted proximally after advancement into bore 104, thereby securing the plug portion to the bone anchor member.

Referring now to FIGS. 3B-3C, further features of plug portion 110 are described. Plug portion 110 comprises first and second passages 118 and 120. First and second suture ends 32a and 32b preferably are coupled to plug portion 110 of apparatus 100 in a manner similar to that described in FIG. 2C hereinabove. Specifically, in the embodiment of FIGS. 3A-3C, first end 32a of suture 30 is disposed through first passage 118. After exiting through first passage 118, first end 32a then transitions into loop portion 34a, forms loop 34, and transitions into loop portion 34b (see FIGS. 2A-2C). Loop portion 34b transitions into second end 32b, which extends through second passage 120 of FIGS. 3A-3C.

If desired, passages 118 and 120 of FIGS. 3A-3C may comprise cleated members 74, as described hereinabove with respect to FIG. 2C. If cleated members 74 are employed, then tissue T may be secured to bone B by individually tensioning first and second ends 32a and 32b of suture 30, as described hereinabove with respect to FIG. 2C.

Plug portion 110 preferably comprises one or more guide channels 125 disposed in a lateral surface of plug body 113. Guide channel 125 preferably is substantially similar to guide channels 50 and 52 of FIG. 2C. In FIG. 3C, guide channel 125 is configured to permit retraction of first and second suture ends 32a and 32b when plug portion 110 is secured within bore 104 by providing a clearance between the plug portion and the bone anchor member.

Alternatively, in the embodiment of FIGS. 3A-3C, passages 118 and 120 may be substantially smooth passages, such that cleated members 74 are not employed. In this case, passages 118 and 120 permit substantially unimpeded movement of suture 30 through the passages. In operation, a physician may individually tension suture ends 32a and 32b prior to insertion of plug portion 110 into bone anchor member 102. When a physician deems that tissue T is appropriately secured to bone B, then plug portion 110 is forced into bore 104 of bone anchor member 102. This causes suture ends 32a and 32b to be sandwiched between plug portion 110 and bone anchor member 102 when guide channels 125 are not present. Accordingly, the suture is secured between the two portions using a force fit.

Referring now to FIGS. 4A-4C, an alternative embodiment of the present invention is described. In FIG. 4A, apparatus 140 comprises bone anchor member 142 and plug portion 150. Bone anchor member 142 comprises main body 143 having bore 144 disposed therein, as depicted in FIG. 4A. Further, main body 143 of bone anchor member 142 comprises exterior cleated members 146 and interior cleated members 145. Exterior cleated members 146 are configured to be inserted into hole H of bone B (see FIG. 1) using a force fit, as described hereinabove.

Plug portion 150 comprises main body 153, which preferably has a substantially cylindrical shape and smooth exterior surface 156. Taper 157 preferably is disposed at a distal region of main body 153, as shown in FIG. 4A.

Suture 30 having first and second ends 32a and 32b is coupled to plug portion 150 of apparatus 140, preferably in a manner described hereinabove with respect to FIGS. 2-3. More specifically, suture 30 passes through first and second passages 158 and 160 of plug portion 150 in a manner described hereinabove, e.g., with respect to FIG. 2C.

In operation, bone anchor member 142 is advanced into hole H (see FIG. 1). Exterior cleated members 146 of bone anchor member 142 permit one-way movement of the bone anchor member into the hole.

In a next step, plug portion 150 then is inserted into bore 144 of bone anchor member 142. Exterior surface 156 of plug portion 150 preferably has an outer diameter that is slightly larger than an inner diameter of bore 144. Accordingly, when plug portion 150 is urged distally, a force fit is achieved to secure plug portion 150 within the bore of bone anchor member 142.

Taper 157 of plug portion 150 facilitates the distal advancement of the plug portion with respect to bone anchor member 142. Further, interior cleated members 145 are configured to permit advancement of plug portion 110 into bore 144 in a distal direction only.

First and second suture ends 32a and 32b may be coupled to plug portion 150 in a manner described hereinabove with respect to FIGS. 3A-3C. Specifically, in the embodiment of FIGS. 4A-4C, first end 32a of suture 30 is disposed through first passage 158, then forms a loop that is threaded through tissue T, and second end 32b of suture 30 then extends through second passage 160.

First and second passages 158 and 160 of FIGS. 4A-4C may comprise cleated members 74 of FIG. 2C. If cleated members 74 are employed, then tissue T may be secured to bone B by individually tensioning first and second ends 32a and 32b of suture 30. Cleated members 74 permit incremental tensioning of each suture end, and serve to lock the suture ends within their respective passages 158 and 160, as generally set forth hereinabove with respect to FIG. 2C.

Plug portion 150 preferably comprises one or more guide channels 165 disposed in a lateral surface of plug body 153, as shown in FIG. 4C. Guide channel 165 preferably is substantially similar to guide channel 50 of FIG. 2C, and is configured to permit retraction of first and second suture ends 32a and 32b when plug portion 150 is secured within bore 144.

Alternatively, in the embodiment of FIGS. 4A-4C, passages 158 and 160 may be substantially smooth passages, such that cleated members 74 are not employed and guide channels 165 are not present. In this case, a physician may individually tension suture ends 32a and 32b prior to insertion of plug portion 150 into bone anchor member 142. When a physician deems that tissue T is appropriately secured to bone B, then plug portion 150 is forced into bore 144 of bone anchor member 142. This causes suture ends 32a and 32b to be sandwiched between plug portion 150 and bone anchor member 142. Accordingly, the suture is secured between the two portions using a force fit.

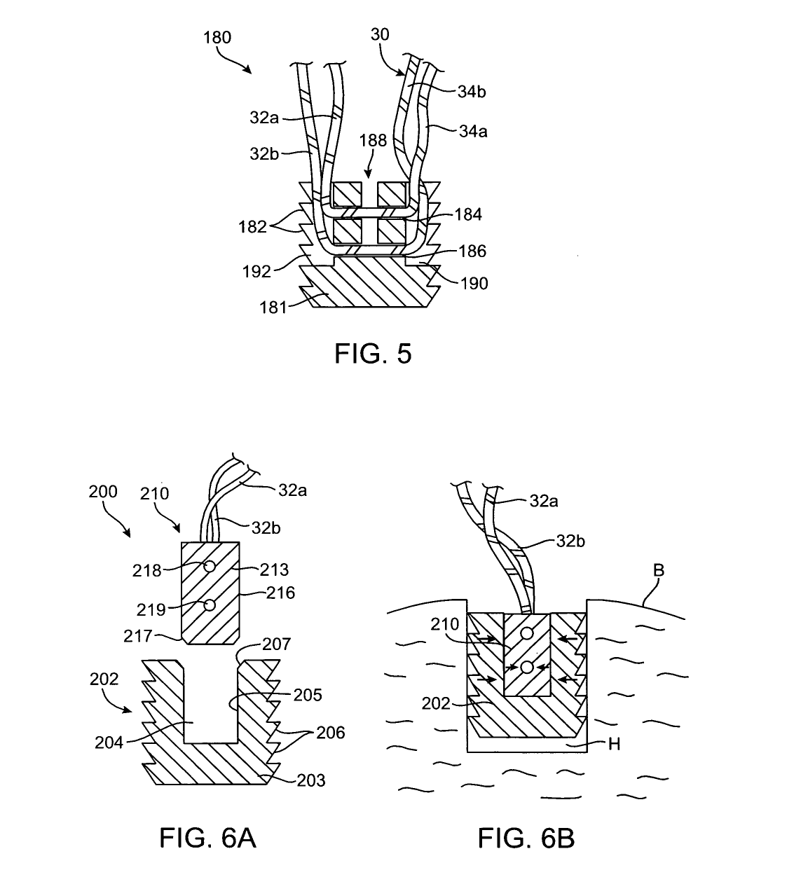

Referring now to FIG. 5, a further alternative embodiment of the present invention is described. In FIG. 5, bone anchor member 180 is similar to bone anchor member 20 of FIGS. 2A-2C, except as noted hereinbelow. Cleated members 182 of bone anchor member 180 preferably are similar to cleated members 42 of bone anchor member 20, as described hereinabove, and facilitate anchoring of bone anchor member 180 within hole H. Further, guide channels 190 and 192 preferably are similar to guide channels 50 and 52 of FIGS. 2A-2C.

Unlike the embodiments described hereinabove, bone anchor member 180 comprises at least one adhesive delivery channel 188, which is provided within main body 181 as shown in FIG. 5. Adhesive delivery channel 188 may be formed by drilling a hole into an upper surface of main body 181, such that the hole extends through first passage 184 and second passage 186. As will be apparent to one skilled in the art, however, channel 188 may be formed using other known techniques.

In the embodiment of FIG. 5, first and second passages 184 and 186 may comprise cleated members 74 of FIG. 2C, thereby permitting one-way movement of suture ends 32a and 32b through the passages. Alternatively, in the embodiment of FIG. 5, passages 184 and 186 may comprise substantially smooth inner surfaces that permit movement of suture 30 through the passages in either direction.

After bone anchor member 180 is secured in hole H of bone B, a physician may approximate the positioning of tissue T with respect to bone B by individually tensioning first and second ends 32a and 32b of suture 30, as described hereinabove. When the suture ends are tensioned as desired, an adhesive is delivered to adhesive delivery channel 188, preferably using a needle-like tube (not shown) disposed within a working cannula (not shown). The needle-like tube preferably has a distal opening that may be placed in close proximity to, or within, adhesive delivery channel 188 to deliver an adhesive thereto.

The adhesive flows distally through adhesive delivery channel 188 and into portions of first and second passages 184 and 186. The adhesive contacts portions of suture 30 that extend through corresponding regions of first and second passages 184 and 186, thereby locking the suture in place. As will be apparent to one skilled in the art, although one adhesive delivery channel 188 is depicted in FIG. 5, multiple adhesive delivery channels may be employed to secure the suture, irrespective of whether cleated members 74 are employed.

Referring now to FIGS. 6A-6B, yet another alternative embodiment of the present invention is described. In FIG. 6A, apparatus 200 comprises bone anchor member 202 and plug portion 210. Apparatus 200 is similar to apparatus 140 of FIGS. 4A-4C, except as noted below.

Bone anchor member 202 comprises main body 203 having bore 204 disposed therein, as depicted in FIG. 6A. Further, main body 203 of bone anchor member 202 comprises exterior cleated members 206, which are configured to be inserted into hole H of bone B (see FIG. 1) using a force fit, as described hereinabove.

Plug portion 210 preferably comprises a substantially cylindrical shape and comprises main body 213 having substantially smooth exterior surface 216. Further, taper 217 preferably is disposed at a distal region of main body 213, as shown in FIG. 6A.

Suture 30 having first and second ends 32a and 32b is coupled to plug portion 210, preferably in a manner described hereinabove with respect to FIGS. 2-4. More preferably, suture 30 passes through first and second passages 218 and 219 of plug portion 210 in a manner described hereinabove with respect to FIG. 2C.

Main body 213 of plug portion 210 has an outer diameter that is slightly larger than an inner diameter of bore 204. The diameters are selected such that main body 213 of plug portion 210 may be distally advanced into bore 204 when forced distally. Taper 207 of bone anchor member 202 is configured to facilitate advancement of plug portion 210 into bore 204.

In operation, bone anchor member 202 is secured within hole H when the bone anchor member is distally advanced into the hole, as depicted in FIG. 6B. Exterior cleated members 206 of bone anchor member 202 permit oneway movement of the bone anchor member into hole H.

In a next step, plug portion 210 is advanced distally into bore 204 of bone anchor member 202 and secured therein using a force fit, as described hereinabove. At this time, surrounding regions of bone B will apply a compressive force upon bone anchor member 202, as indicated by the larger directional arrows in FIG. 6B. This compressive force upon bone anchor member 202 in turn causes compression upon plug portion 210, as indicated by the smaller directional arrows in FIG. 6B, thereby securely retaining the plug portion within bore 204.

In the embodiment of FIGS. 6A-6B, passages 218 and 219 may comprises cleated members 74 as described hereinabove with respect to FIG. 2C, as a means for locking suture 30. Alternatively, passages 218 and 219 may comprise substantially smooth interior surfaces that permit advancement of suture 30 in either direction.

In either embodiment, it may be desirable to approximate the positioning of tissue T (not shown in FIG. 6B) to bone B by individually tensioning suture ends 32a and 32b prior to insertion of plug portion 210 into bone anchor member 202. In a preferred embodiment of the method, the tissue position is approximated when passage 219 is disposed just above bore 204. Once the desired positioning of the tissue is achieved, plug portion 210 is advanced distally into bore 204, thereby locking the suture. Specifically, the suture will be sandwiched between exterior surface 216 of plug portion 210 and inner wall 205 of bone anchor member 202.

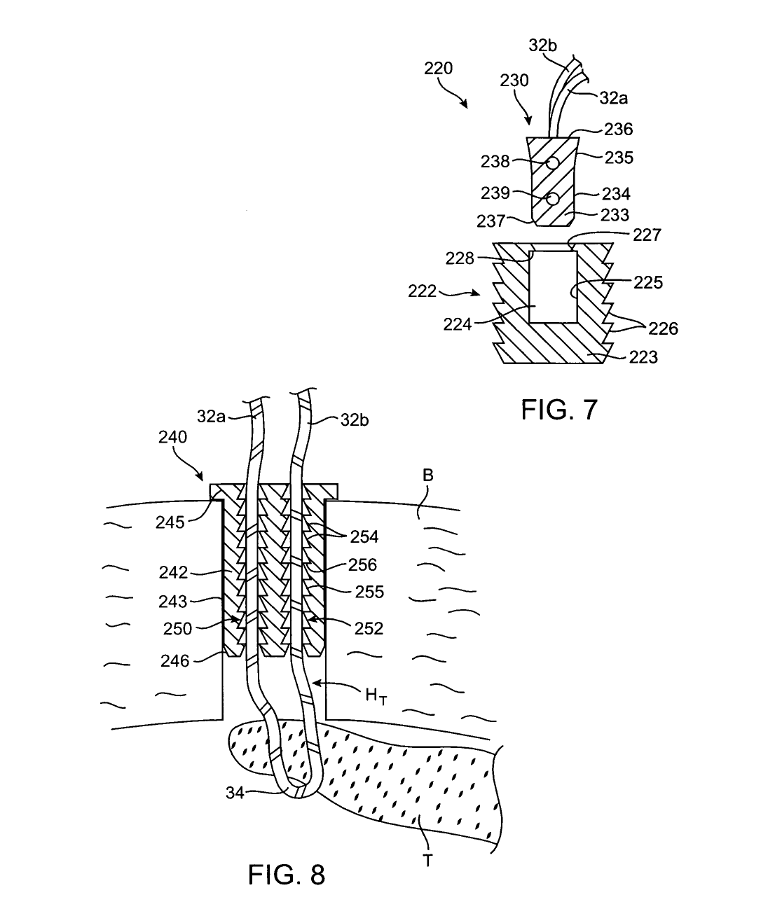

Referring now to FIG. 7, a further alternative embodiment of the present invention is described. In FIG. 7, apparatus 220 comprises bone anchor member 222 and plug portion 230. Apparatus 220 is similar to apparatus 200 of FIGS. 6A-6B, except as noted below.

Bone anchor member 222 comprises main body 223 having bore 224 disposed therein, as depicted in FIG. 7. Further, main body 223 comprises exterior cleated members 226, which are configured to be inserted into hole H of bone B (see FIG. 1) using a force fit, as described hereinabove. Unlike previous embodiments, bone anchor member 222 comprises a proximal protrusion having inward taper 227. Proximal stop 228 is formed between inward taper 227 and inner wall 225 of bone anchor member 222, as shown in FIG. 7.

Plug portion 230 preferably comprises main body 233 having proximal region 235, central region 234 and tapered distal region 237. Tapered distal region 237 is sized to pass through taper 227 of bone anchor member 222 when a distally-directed force is applied to plug portion 230. When further force is applied, central region 234 of plug portion 230 is advanced into bore 224 via taper 227. Finally, when yet further force is applied to plug portion 230, proximal region 235 will be advanced past taper 227. Once proximal region 235 is fully inserted into bore 224, proximal stop 228 is configured to abut proximal edge 236 of plug portion 230, thereby securing the plug portion within bone anchor member 222.

As will be apparent to one skilled in the art, apparatus 220 of FIG. 7 may comprise any other features described hereinabove with respect to the embodiments of FIGS. 2-6. For example, passages 238 and 239 may comprise cleated members 74 of FIG. 2C, or alternatively may comprise substantially smooth interior surfaces. Further, the operation of apparatus 220 preferably is substantially similar to the methods described hereinabove with respect to the embodiments of FIGS. 2-6.

Referring now to FIG. 8, a further alternative embodiment of the present invention is described. In FIG. 8, bone anchor 240 is similar to bone anchor member 20 of FIGS. 1-2, but is configured for use in applications where through hole H_{T} is employed. For example, bone B may be a thin bone, such that it is possible to arthroscopically operate from both sides of the bone.

Bone anchor 240 comprises main body 242 having proximal and distal ends, flange 245 disposed at the proximal end and taper 246 formed at the distal end. Main body 242 further comprises exterior surface 243 disposed between flange 245 and taper 246, as shown in FIG. 8.

Bone anchor 240 further comprises first and second passages 250 and 252, each having a plurality of cleated members 254, as shown in FIG. 8. Each of the cleated members comprises angled sections 255 and substantially orthogonal sections 256, which are disposed adjacent one another thereby forming a cleated shape, as described hereinabove with respect to cleated members 74 of FIG. 2C.

In operation, a central region of suture 30 can be looped through tissue T first, such that free ends 32a and 32b extend from the tissue. Free end 32a then is threaded through first passage 250 in a proximal direction, while free end 32b is threaded through second passage 252, also in a proximal direction. The suture may be threaded through passages 250 and 252 and tissue T by arthroscopically operating on one or both sides of bone B.

As will be apparent to one skilled in the art, suture 30 may be coupled between tissue T and bone anchor 240 using other arthroscopic threading techniques, so long as the suture ultimately is situated in a manner depicted in FIG. 8.

Once the suture is threaded as shown in FIG. 8, a physician may proximally retract first and second suture ends 32a and 32b, one at a time, to approximate the positioning of tissue T with respect to bone B. As the suture ends are tensioned, flange 245, which has an outer diameter larger than the diameter of through hole H_{T}, abuts bone B. The system becomes tensioned because flange 245 and tissue T are drawn against the bone from opposing directions.

In accordance with one aspect of the present invention, cleated passages 250 and 252 are configured to permit one-way movement of first and second suture ends 32a and 32b, respectively. For example, when first end 32a is pulled in a proximal direction by a physician, angled sections 255 permit movement of that particular suture end in the proximal direction. However, a physician cannot distally advance suture end 32a within passage 250. Advantageously, the use of two separate passages allows the surgeon to tension each end of the suture separately, which is often desirable when tissue T is torn irregularly.

As will be apparent to one skilled in the art, the methods described in FIG. 8 may be accomplished using a separate bone anchor member and plug portion. For example, the principles of the embodiments in FIGS. 3-4 and 6-7, in which separate bone anchor and plug portions are employed, may be implemented in lieu of one-piece bone anchor 240.

Further, the suture securing methods described in FIG. 8 may be accomplished using substantially smooth passages 250 and 252. Where substantially smooth passages are employed, an interference fit or adhesive may be employed in lieu of the cleated passages to facilitate securing of the suture. The interference fit or adhesive may be used, for example, as described hereinabove with respect to the embodiments of FIGS. 3-7.

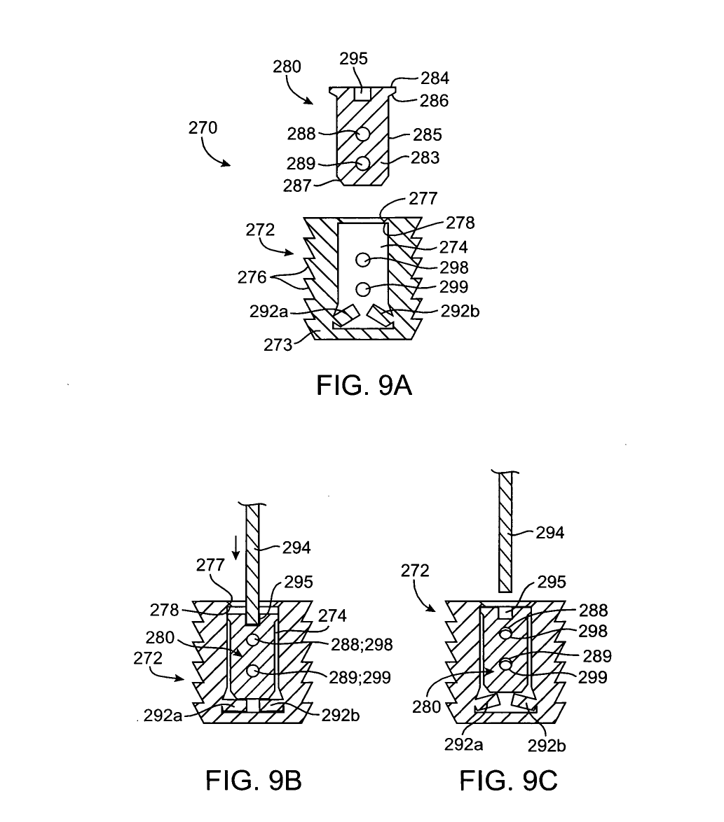

Referring now to FIGS. 9A-9C, still a further alternative embodiment of the present invention is described. In FIG. 9A, apparatus 270 comprises bone anchor member 272 and plug portion 280.

Bone anchor member 272 comprises main body 273 having bore 274 disposed therein, as depicted in FIG. 9A. Further, main body 273 comprises exterior cleated members 276, which are configured to be inserted into hole H of bone B (see FIG. 1) using a force fit, as described hereinabove. Like the embodiment of FIG. 7, bone anchor member 272 comprises a proximal protrusion having inward taper 277. Proximal stop 278 is formed between inward taper 277 and an inner wall of bone anchor member 272, as shown in FIG. 9A.

Bone anchor member 272 further comprises first and second spring elements 292a and 292b, which are disposed at a distal region of bore 274. First and second spring members 292a and 292b may be integrally formed with bone anchor body 273, as depicted in FIG. 9A, or may be separate elements coupled to body 273. As will be described in further detail hereinbelow, first and second spring elements 292a and 292b may be deformed to accommodate plug portion 280 within bore 274, and also to enable locking and unlocking of a suture (not shown in FIGS. 9A-9C) used in conjunction with apparatus 270. As will be apparent to one skilled in the art, one or more spring elements may be employed.

First and second passages 298 and 299 extend laterally through main body 273 of bone anchor member 272, as depicted in FIG. 9A. First and second passages 298 and 299 are configured to be selectively aligned with first and second passages 288 and 289 of plug portion 280, for the purposes described hereinafter.

Referring still to FIG. 9A, plug portion 280 of apparatus 270 preferably comprises main body 283 having proximal and distal ends. The proximal end comprises flange 284. Taper 286 is disposed between flange 284 and main body 283, as shown in FIG. 9A. Further, distal taper 287 is disposed at the distal end of plug portion 280.

Plug portion 280 further comprises first and second passages 288 and 289, which extend laterally through main body 283, as shown in FIG. 9A. In the embodiment of FIGS. 9A-9C, first and second passages 288 and 289 preferably comprise a substantially smooth interior surfaces.

Referring now to FIG. 9B, in a first step, plug portion 280 is inserted into bore 274 of bone anchor member 272, preferably using insertion tool 294. Specifically, when an appropriate force is applied to plug portion 280, tapered distal end 287 is configured to pass through taper 277 of bone anchor member 272. When further force is applied, a central region of plug portion 280 is advanced into bore 274 via taper 277. Finally, when yet further force is applied to plug portion 280, the proximal region having taper 286 and flange 284 will be advanced past taper 277.

When plug portion 280 is fully inserted into bore 274, first and second spring elements 292a and 292b will be inclined to urge plug portion 280 in a proximal direction, such that flange 284 will abut proximal stop 278 (see FIG. 9C). However, when a sufficient distally-directed force is applied to plug portion 280, e.g., using insertion tool 294, first and second spring elements may be deformed distally, as shown in FIG. 9B.

Insertion tool 294 may be a rod or other substantially rigid member configured to transfer a distally-directed force from a physician to plug portion 290. In a preferred embodiment, insertion tool 294 is configured to engage mating slot 295, as shown in FIG. 9B.

The provision of a distally-directed force acting on plug portion 280 causes first and second passages 288 and 289 to become substantially aligned with first and second passages 298 and 299 of bone anchor member 27, respectively, as shown in FIG. 9B. At this time, suture 30 is threaded through aligned first passages 288 and 298. The suture then is threaded through tissue T, as described hereinabove, and then threaded back through aligned second passages 289 and 299. In effect, first suture end 32a extends through first passages 288 and 298, while second suture end 32b extends through second passages 289 and 299.

Once the suture is coupled to apparatus 270 in this manner, apparatus 270 is inserted into hole H of bone B under arthroscopic guidance. Cleated members 276 secure apparatus 270 within hole H, as described hereinabove. At this time, first and second suture ends 32a and 32b will extend outside of the arthroscopic site for manipulation by a physician.

A physician may selectively tension first and second suture ends 32a and 32b to approximate the positioning of tissue T with respect to bone B when first and second passages 288 and 289 are aligned with first and second passages 298 and 299, respectively. During tensioning of the suture ends, insertion tool 294 urges plug portion distally to cause the passages to align, as shown in FIG. 9B.

When a desired positioning of tissue T is achieved, the force applied to plug portion 280 is removed, e.g., by proximally retracting insertion tool 294, as shown in FIG. 9C. At this time, first and second spring elements 292a and 292b are inclined to bias proximally, thereby urging flange 284 of plug portion 280 against proximal stop 278 of bone anchor member 272. This movement of plug portion 280 with respect to bone anchor member 272 causes a misalignment between first passage 288 of plug portion 280 and first passage 298 of bone anchor member 272. Also, a misalignment occurs between second passages 289 and 299. Accordingly, the misalignments cause first suture end 32a to become pinched between first passages 288 and 298, while second suture end 32b is pinched between second passages 289 and 299. These misalignments lock the suture in place.

If it becomes necessary to adjust the positioning of tissue T with respect to bone B during the procedure, then insertion tool 294 may be inserted into mating slot 295, as shown in FIG. 9B, to urge plug portion 280 distally. As described hereinabove, when first and second passages of plug portion 280 and bone anchor member 272 are aligned (see FIG. 9B), a physician may manipulate suture ends 32a and 32b to adjust the positioning of tissue T.

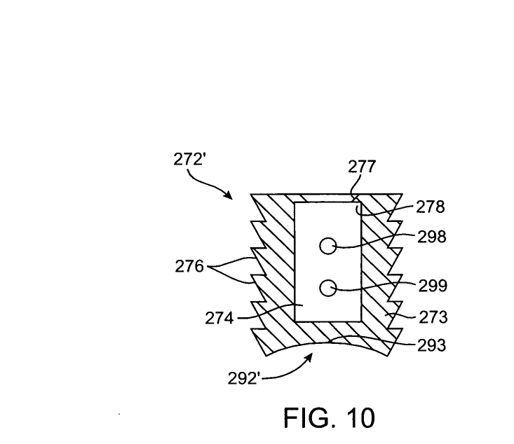

Referring now to FIG. 10, an alternative embodiment of the invention of FIGS. 9A-9C is described. In FIG. 10, alternative bone anchor member 272′ comprises spring element 292′ disposed at a distal end of main body 273. Spring element 292′ comprises a distally concave configuration having a central region 293, as shown in FIG. 10.

Alternative bone anchor member 272′ is used in conjunction with plug portion 280 in a manner similar to that described hereinabove with respect to FIGS. 9A-9C. Specifically, after plug portion 280 is inserted into bore 274, the provision of a further distally-directed force acting on plug portion 280 will cause central region 293 of spring element 292′ to be deformed in a distal direction. When the central region of spring element 292′ is deformed distally, first and second passages 288 and 289 of plug portion 280 are substantially aligned with first and second passages 298 and 299 of bone anchor member 272′, respectively. In this state, first suture end 32a may move substantially unimpeded through aligned first passages 288 and 298, while second suture end 32b may move through aligned second passages 289 and 299, respectively, as described hereinabove with respect to FIG. 9B.

When a desired positioning of tissue T is achieved, the force imposed upon plug portion 280 is removed, e.g., by proximally retracting insertion tool 294, as described in FIG. 9C. At this time, central region 293 of spring elements 292′ will return in a proximal direction to its preferred orientation. This causes flange 284 of plug portion 280 to be urged against proximal stop 278 of bone anchor member 272′. As described hereinabove, the movement of plug portion 280 with respect to bone anchor member 272′ causes a misalignment between first passages 288 and 298, and also a misalignment between second passages 289 and 299. These misalignments pinch suture ends 32a and 32b to lock the suture in place.

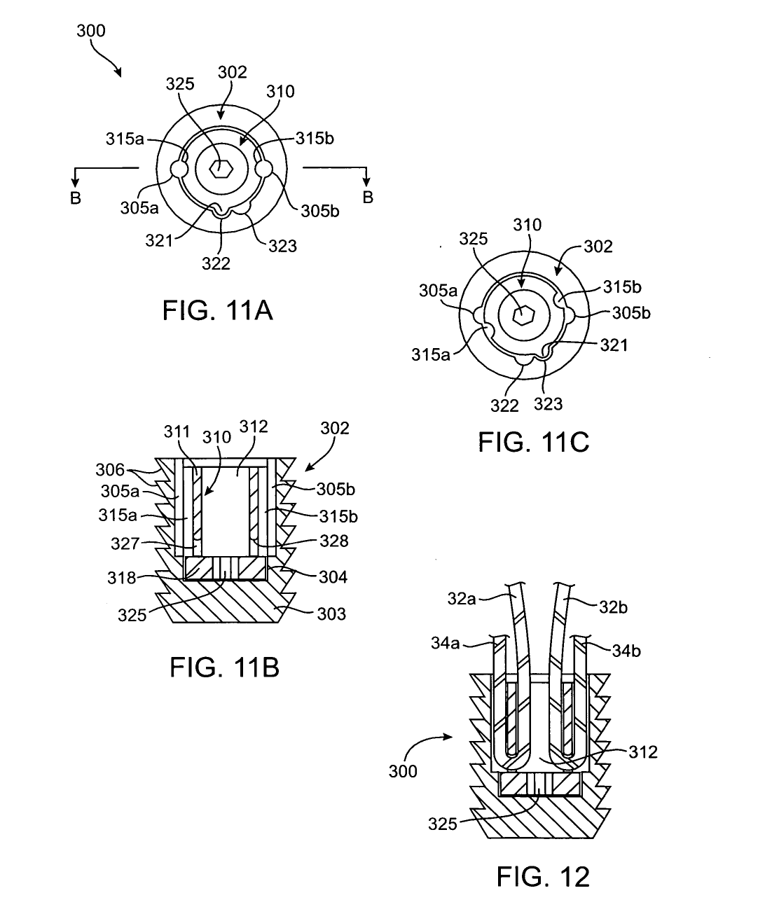

Referring now to FIGS. 11-12, a further alternative embodiment of the present invention is described. Apparatus 300 comprises bone anchor member 302 and plug portion 310, as shown in FIGS. 11A-11B. Bone anchor member 302 is similar to the bone anchor members described hereinabove and comprises main body 303 having plurality of cleated members 306, which are configured to anchor plug portion 302 within hole H of bone B (see FIG. 1). Bone anchor member 302 further comprises central bore 304, which is configured to receive plug portion 310, as described hereinbelow.

Plug portion 310 of apparatus 300 comprises main body 311 having distal region 318 and central bore 312, as shown in FIG. 11B. Main body 311 has an outer diameter that is slightly smaller than an inner diameter of bore 304. Accordingly, plug portion 310 is configured for circumferential rotation within bore 304 of bone anchor member 302, as described hereinbelow.

Bone anchor member 302 further comprises first and second semi-circular channels 305a and 305b, which preferably are formed at diametrically opposing surfaces of main body 303, as shown in FIGS. 11A-11B. Further, plug portion 310 comprises first and second semi-circular channels 315a and 315b, which preferably are formed at diametrically opposing surfaces on main body 311, as shown in FIGS. 11A-11B.

Apparatus 300 also comprises actuation knob 321, which is disposed on an outer surface of plug portion 310, as shown in FIG. 11A. Actuation knob 321 is configured to be disposed within first recess 322 of bone anchor member 302 in an unlocked state, and disposed within second recess 323 in a locked state, as described in further detail hereinbelow.

When actuation knob 321 is disposed within first recess 322, first and second semi-circular channels 305a and 305b of bone anchor member 302 are aligned with first and second semi-circular channels 315a and 315b of plug portion 310, respectively, thereby forming first and second circular channels, as shown in FIGS. 11A-11B.

When actuation knob 321 is disposed within second recess 323, first and second semi-circular channels 305a and 305b of bone anchor member 302 are not aligned with corresponding channels 315a and 315b of plug portion 310, as shown in FIG. 11C.

In operation, suture 30 preferably is coupled to apparatus 300 in a manner shown in FIG. 12. Specifically, first suture end 32a extends through central bore 312 of plug portion 310. First suture end 32a passes through aperture 327 in plug portion 310 (see FIG. 11B) and transitions into loop portion 34a. Loop portion 34a is threaded through the first circular channel formed by semi-circular channels 305a and 315a, as shown in FIG. 12.

Loop portion 34a then is threaded through tissue T and transitions into loop portion 34b. Loop portion 34b is threaded through the second circular channel formed by semi-circular channels 305b and 315b, as shown in FIG. 12. Loop portion 34b passes through a second aperture 327 and transitions into second suture end 32b. Second suture end 32b extends through central bore 312 of plug portion 310, as shown in FIG. 12.

In accordance with one aspect of the present invention, a physician may selectively tension first and second suture ends 32a and 32b when actuation knob 322 is disposed within first recess 322, as shown in FIGS. 11A-11B. This is because first and second semi-circular channels 305a and 305b of bone anchor member 302 are aligned with first and second semi-circular channels 315a and 315b of plug portion 310, respectively, to form the first and second circular channels through which the suture can freely pass.

It should be noted that, as first and second ends 32a and 32b are individually tensioned, rounded edges 328 of plug portion 310 (see FIG. 11B) serve to reduce the shear stresses imposed upon the suture ends as they pass through apertures 327.

When a physician desires to lock the suture in place, plug portion 310 is rotated with respect to bone anchor member 302 to cause actuation knob 321 to be advanced into second recess 323. The rotation of plug portion 310 may be achieved by inserting an actuation tool such as a hexagonal key (not shown) into mating slot 325. Once knob 321 is secured within second recess 323, as shown in FIG. 11C, the suture will be locked in place because the misaligned semi-circular channels pinch the first and second ends of the suture.

Advantageously, if a physician desired to tweak the positioning of tissue T with respect to bone B after the suture has been locked, then a physician simply needs to insert the actuation tool into mating slot 325 to cause knob 322 to rotate in an opposing direction into first recess 322. As described above, this forms two fully circular channels through which the suture may be advanced or retracted to facilitate positioning of the tissue with respect to the bone.

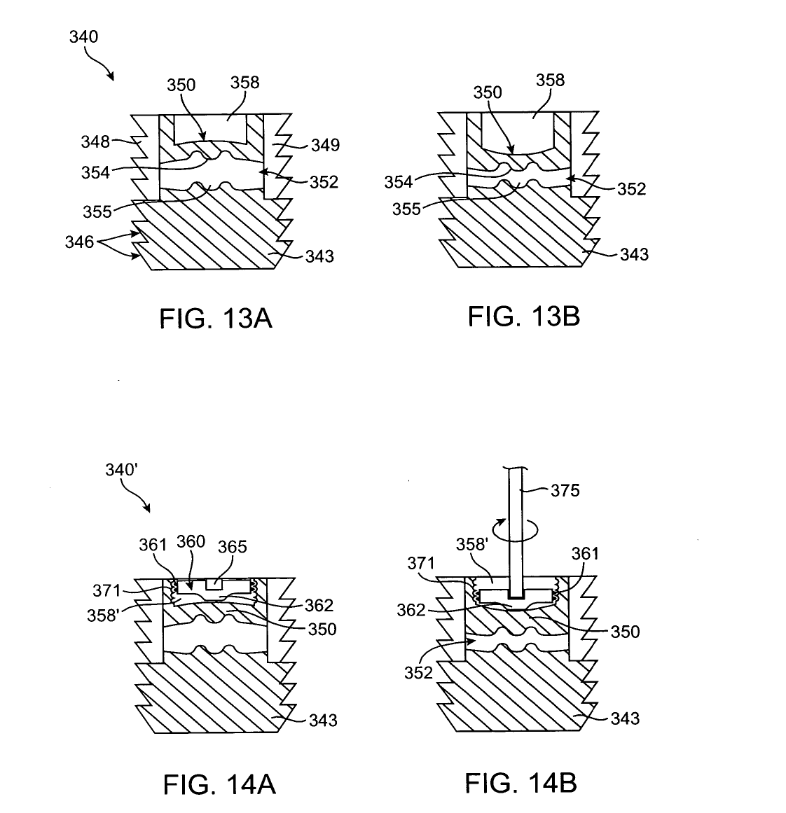

Referring now to FIGS. 13A-13B, a further alternative embodiment of the present invention is described. In FIG. 13A, bone anchor member 340 comprises main body 343 having proximal and distal regions. Bone anchor member 340 preferably comprises plurality of cleated members 346, and further comprises opposing guide channels 348 and 349, which preferably are similar to guide channels 50 and 52 of FIG. 2C.

Bone anchor member 340 further comprises at least one passage 352, which extends laterally through main body 343, and further comprises flexible member 350, which is disposed proximal to passage 352, as shown in FIGS. 13A-13B. Flexible member 350 has a preferred relaxed configuration in which it assumes a convex shape, i.e., bowed away from passage 352. In the relaxed configuration, shown in FIG. 13A, there is sufficient clearance between flexible member 350 and passage 352 to permit suture 30 to move substantially unimpeded through the passage.

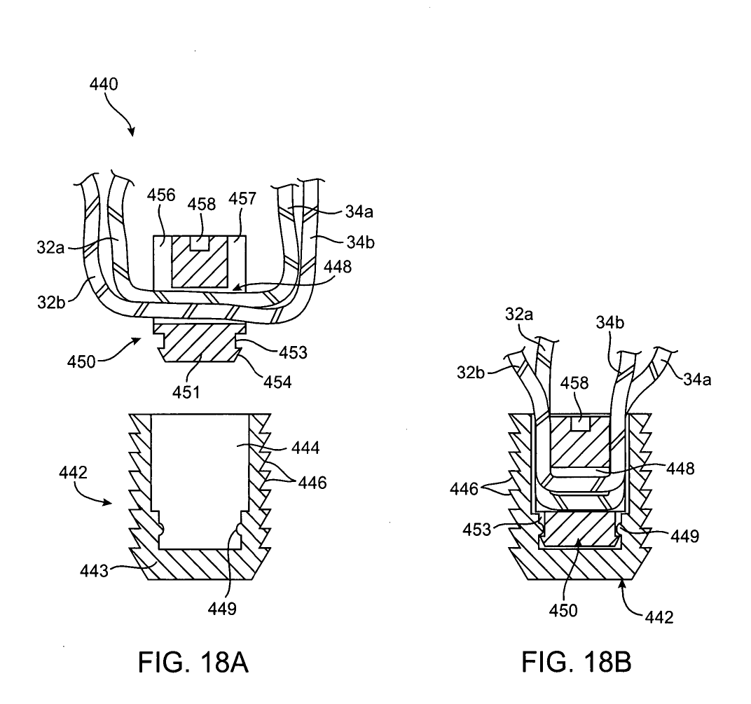

In operation, suture 30 may be coupled between apparatus 340 and tissue T, for example, in a manner described hereinbelow with respect to FIG. 18A. Specifically, before bone anchor member 340 is inserted into hole H in bone B, first suture end 32a is passed through passage 352. The first suture end then becomes loop portion 34a, which is threaded through tissue T, as described hereinabove. Loop portion 34a extends through the tissue to become loop portion 34b. Loop portion 34b passes back through passage 352 and becomes second suture end 32b. First and second suture ends 32a and 32b extend outside of the arthroscopic site and may be individually tensioned by a physician.

After suture 30 is coupled to apparatus 340 and tissue T, bone anchor member 340 is advanced distally into hole H of bone B (see FIG. 1), whereby cleated members 346 serve to anchor the device in hole H. As described above, a physician then may individually tension first and second suture ends 32a and 32b to approximate the positioning of tissue T with respect to bone B. During this time, no external forces are applied to flexible member 350, thereby permitting movement of the suture within passage 352.

Once a desired tissue positioning is achieved, the suture may be locked in place by apply a distally-directed force upon flexible member 350, as depicted in FIG. 13B. Flexible member 350 preferably assumes a concave shape in which distal knob 354 is urged towards corresponding pocket 355 in bone anchor member 342. The distally-directed force locks the suture in place by pinching the suture and inhibiting its movement within passage 352.

As will be apparent to one skilled in the art, any number of mechanisms may be employed to apply a distally-directed force upon flexible member 350, and further, to lock the flexible member in the concave position depicted in FIG. 13B. For example, a plug may be inserted into bore 358, and then wedged against flexible member 350 to hold the flexible member in place. Alternatively, bone anchor member 340 may comprise taper 277 and proximal stop 278 (see FIG. 9A) such that the plug will remain in place within bore 358. In either case, the plug will serve to apply a compressive force to hold the suture in the locked state.

Alternatively, the flexible member may be “bi-stable,” such that the flexible member has only two stable states. In the first state, the flexible member is positioned as shown in FIG. 13A. When a sufficient distally-directed force is applied, the flexible member is configured to “snap” from the first state into a second state, as shown in FIG. 13B. There are no stable positions between the first and second state. Accordingly, the flexible member is either provided in a locked or unlocked state. Means for applying a proximally-directed force to the flexible member may be used to cause the flexible member to snap from the second state, shown in FIG. 13B, to the first state, shown in FIG. 13A, thereby unlocking the device.

In an alternative embodiment, a threaded member may be used to hold the suture in a locked state. As shown in FIGS. 14A-14B, threaded cap 360 has exterior thread 361, which is adapted to engage grooved interior section 371 of bore 358′. In a preferred embodiment, threaded cap 360 further comprises a proximal region having mating slot 365 and a distal region having distal protrusion 362.

In an unlocked state, threaded cap 360 is situated proximally within bore 358′, as shown in FIG. 14A. Once a physician wishes to lock the suture in place, locking tool 375 may be inserted into mating slot 365 and then rotated clockwise to advance threaded cap in a distal direction, in a manner similar to tightening a screw. This causes a distal region of threaded cap 360, and preferably, distal protrusion 362, to urge flexible member 350 distally, thereby impinging upon a suture length disposed through passage 352. This effectively locks the suture in place.

If a physician subsequently desires to re-adjust the suture, then locking tool 375 can be rotated counterclockwise within mating slot 365 to proximally retract the threaded cap. This will remove the forces imposed upon the suture, as depicted in FIG. 14A.

In the embodiment of FIGS. 14A-14B, it will be apparent to one skilled in the art that flexible member 350 may be omitted entirely. In this case, threaded cap 360 will directly pinch the suture in passage 352 to lock the suture in place.

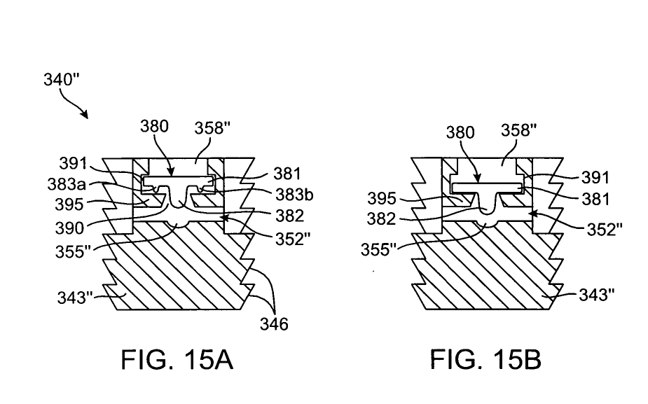

Referring now to FIGS. 15A-15B, a further alternative embodiment of the bone anchor of FIGS. 13A-13B is described. Operation of bone anchor member 340″ is substantially the same as that of bone anchor 340, with the main exception that locking member 380 is provided in lieu of flexible member 350.

Locking member 380 preferably comprises cylindrical body 381, which is configured to be confined within recess 391 of main body 343″, as shown in FIG. 15A. Locking member 380 further comprises distal protrusion 382, which is configured to extend at least partially through aperture 390 of main body 343″.

First and second support members 383a and 383b are disposed beneath cylindrical body 381, and preferably are formed integrally with locking member 380. As shown in FIG. 15A, the first and second support members 383a and 383b rest on support ledge 395 of main body 343″, thereby elevating locking member 380 within recess 391.

In operation, suture 30 is secured to tissue T and disposed through passage 352″, as described hereinabove with respect to FIGS. 13A-13B. Bone anchor member 340″ then is advanced distally into hole H of bone B (see FIG. 1), such that cleated members 346 anchor the device in hole H.

When locking member 380 is elevated within recess 391, distal protrusion 382 does not substantially extend into passage 352″, thereby permitting movement of the suture within passage 352″. At this time, a physician may individually tension first and second suture ends 32a and 32b to approximate the positioning of tissue T with respect to bone B.

Once a desired positioning is achieved, the suture may be locked in place by any number of techniques that cause first and second support members 383a and 383b to be lowered or eliminated, thereby lowering cylindrical body 381 within recess 391 and urging distal protrusion 382 towards corresponding pocket 355″, as depicted in FIG. 15B. The distally-directed force applied by distal protrusion 382 secures the suture in place.

In one embodiment, first and second support members 383a and 383b may be fused with support ledge 395 of main body 343″. In this embodiment, ultrasonic energy may be delivered to a proximal surface of locking member 380, via bore 358″, using techniques that are known in the art. The provision of ultrasonic energy causes first and second support members 383a and 383b to fuse with support ledge 395, thereby lowering locking device 380 and locking the suture disposed within passage 352″ in place.

In the embodiments of FIGS. 13-15, while only one passage 352 is depicted, it will be apparent to one skilled in the art that a second passage may be provided, e.g., disposed adjacent to the first passage. If two adjacent passages 352 are provided, then the suture can be threaded through the first passage, through tissue T, and threaded back through the second passage.

Further, it will be apparent to one skilled in the art that an adhesive, for example, cyanoacrylate, epoxy, bone cement and so forth, may be employed in conjunction with any of the embodiments described in FIGS. 13-15. Such an adhesive may be used in conjunction with apparatus including, but not limited to, flexible member 350, threaded cap 360, locking member 380, and any associated components.

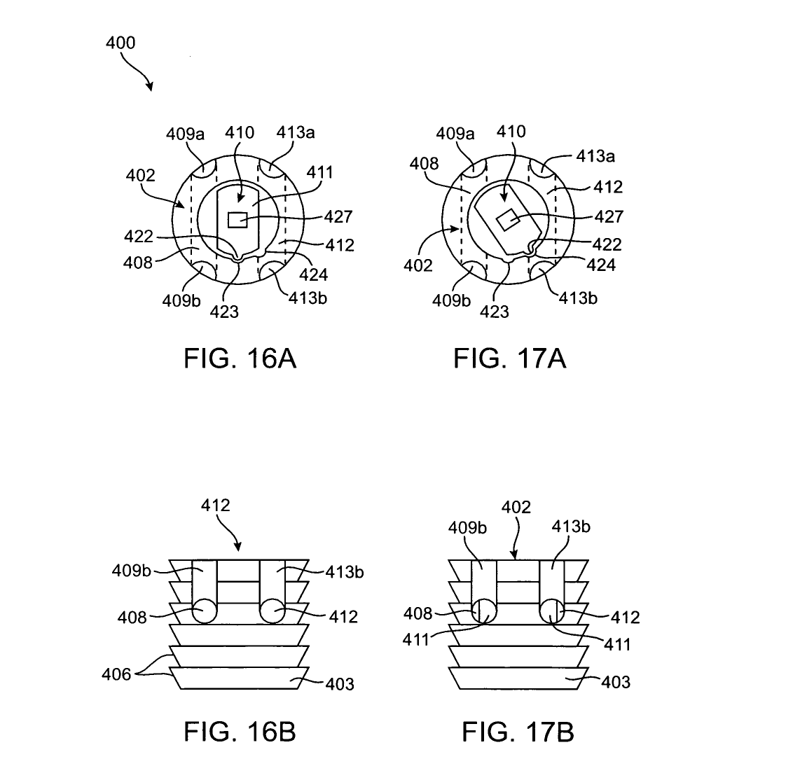

Referring now to FIGS. 16-17, a further alternative embodiment of the present invention is described. In the embodiment of FIGS. 16-17, apparatus 400 comprises bone anchor member 402 and plug portion 410.

Bone anchor member 402 comprises main body 403 having cleated members 406, which are configured to secure bone anchor member 402 in hole H of FIG. 1, as described hereinabove. Further, bone anchor member 402 comprises first and second passages 408 and 412, which extend laterally through main body 403, as shown from a top view in FIG. 16A.

Bone anchor member 402 further preferably comprises guide channels 409a, 409b, 413a and 413b, which are disposed in exterior surfaces of main body 403, as shown in FIGS. 16A-16B. The guide channels preferably are similar to guide channels 50 and 52 of FIGS. 2A-2C, except that four guide channels are employed in the present embodiment.

In use, first suture end 32a passes through guide channel 409a, through passage 408 and through guide channel 409b. The first suture end then transitions into loop 34, which is threaded through tissue T, as described in FIGS. 1-2 hereinabove. Loop 34 of suture 30 then transitions into second suture end 32b. Second suture end 32b passes through guide channel 413b, through passage 412, and through guide channel 413a. Accordingly, the suture is coupled between the tissue and apparatus 400.

Plug portion 410 having main body 411 is configured to be disposed within a central bore of bone anchor member 402. Plug portion 410 comprises actuation knob 422, which is configured to be disposed in first recess 423 of bone anchor member 402 in an unlocked state, and disposed within second recess 424 in a locked state.

In the unlocked state, i.e., when knob 422 is disposed within first recess 423, plug portion 410 is oriented such that main body 411 does not substantially overlap with first and second passages 408 and 412 of bone anchor member 410, as depicted in FIGS. 16A-16B.

In accordance with one aspect of the present invention, a physician may selectively tension first and second ends 32a and 32b of suture 30 when knob 422 is disposed within first recess 423, as shown in FIGS. 16A-16B. This is because first and second passages 408 and 412 provide a substantially unimpeded circular channel within which the suture can pass.

When a physician desires to lock the suture in place, plug portion 410 is rotated to cause knob 422 to be advanced into second recess 424. The rotation of plug portion 410 with respect to bone anchor member 402 may be achieved by inserting an actuation tool such as a rectangular key (not shown) into mating slot 427. Once knob 422 is secured within second recess 424, the suture will be locked in place because main body 411 of plug portion 410 impinges upon passages 408 and 412, as depicted in FIGS. 17A-17B.

Advantageously, if a physician desires to tweak the positioning of tissue T with respect to bone B after the suture is in the locked state, then the physician simply needs to insert the actuation tool into mating slot 427 to cause knob 422 to rotate back into first recess 423 (see FIGS. 16A-16B). This removes the compressive forces imposed upon the suture, such that the first and second ends of the suture may be individually tensioned to facilitate re-positioning of the tissue.

Referring now to FIGS. 18A-18B, yet a further alternative embodiment of the present invention is described. In FIG. 18A, apparatus 440 comprises bone anchor member 442 and plug portion 450. Bone anchor member 442 comprises main body 443 having cleated members 446, which are configured to secure bone anchor member 442 in hole H of FIG. 1, as described hereinabove. Further, bone anchor member 442 comprises central bore 444 and circumferential protrusion 449, which is disposed near a distal end of bore 444, as shown in FIG. 18A.

Plug portion 450 has main body 451 having proximal and distal regions. The proximal region comprises first and second guide channels 456 and 457, which are recessed in opposing lateral surfaces of main body 451. The distal region of main body 451 comprises circumferential recess 453 and distal taper 454, as shown in FIG. 18A. Plug portion 410 also has a central region having passage 448 disposed laterally therethrough, as depicted in FIG. 18A.

Before plug portion 450 is inserted into bore 444, first suture end 32a is passed through passage 448. The first suture end then becomes loop portion 34a, which is threaded through tissue T. Loop portion 34a extends through the tissue to become loop portion 34b. Loop portion 34b passes back through passage 448 and becomes second suture end 32b. First and second suture ends 32a and 32b may be manipulated by a physician, as described in further detail hereinbelow.

Alternatively, as described hereinabove, a central region of suture 30 may be threaded through tissue T, and the free ends of the suture then may be passed through passage 448 in a proximal direction to achieve the suture positioning depicted in FIG. 18A.

In a preferred method of use, bone anchor member 442 is inserted into hole H of bone B before plug portion 450 is inserted into bore 444. Once bone anchor member 442 is securely disposed within hole H, plug portion 450 is positioned slightly above bone anchor member 442, so that passage 448 is proximal to bore 444. At this time, a physician may individually tension first and second suture ends 32a and 32b to approximate the positioning of tissue T with respect to bone B (see FIG. 1).

Once the desired positioning is achieved, the physician advances plug portion 450 distally into bore 444 of bone anchor member 442. An insertion tool, such as insertion tool 294 of FIGS. 9A-9C, may be inserted into mating slot 458 to advance plug portion 450 distally. The provision of a sufficient distally-directed force urges taper 454 over circumferential protrusion 449, thereby locking the plug portion within the bone anchor member, as shown in FIG. 18B.

At this time, first and second suture ends 32a and 32b are compressed within guide channel 456, while suture loop portions 34a and 34b are compressed within guide channel 457, as depicted in FIG. 18B. Guide channels 456 and 457 may be sized to ensure that the suture is completely locked in place when plug portion 450 is inserted into bore 444. Alternatively, guide channels 456 and 457 may be sized to permit incremental adjustments of the suture, such that applying a sufficient tension to free ends 32a and 32b will overcome the frictional forces between the suture, plug portion 450 and bone anchor member 442.

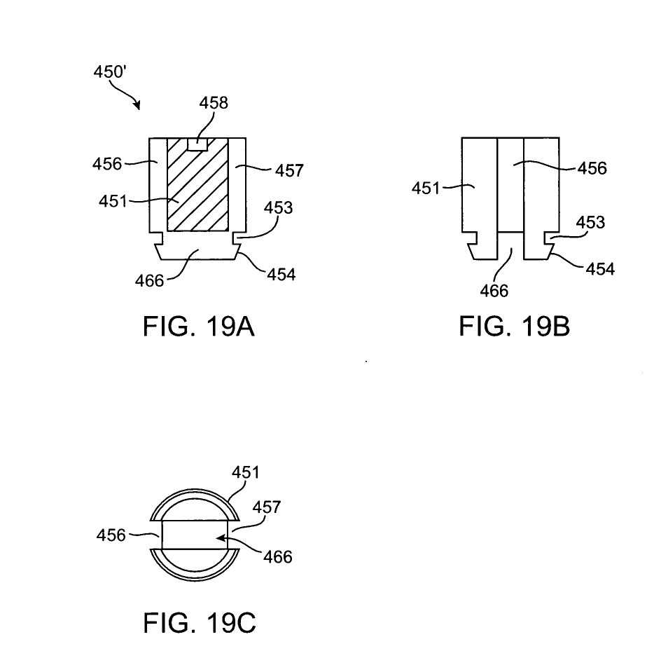

Referring now to FIGS. 19A-19C, an alternative plug portion, which may be used in lieu of plug portion 450 of FIGS. 18A-18B is described. In FIG. 19A, plug portion 450′ comprises distal passage 466, in lieu of passage 448 of FIGS. 18A-18B. Distal passage 466 is formed as a slot recessed in the distal end of main body 451, as shown in FIGS. 19A-19C. Distal passage 466 preferably is in communication with opposing guide channels 456 and 457.

The operation of a bone anchor system using plug portion 450′ is substantially similar to the steps described in FIGS. 18A-18B, with the exception that first and second suture ends 32a and 32b are disposed within distal passage 466. Specifically, in use, the suture ends can be looped around the distal end of plug portion 450′, and need not be inserted by threading through central passage 448. Once the suture ends are looped around the distal end of plug portion 450′ and confined within passage 466, then a physician may hold the suture in place while inserting plug portion 450′ into bone anchor member 442. Once the plug portion is locked into place via circumferential protrusion 449, as described in FIG. 18B, then the suture is compressed between plug portion 450′ and bone anchor member 442.

In each of the embodiments described hereinabove, it will be apparent to those skilled in the art that various means for securing a bone anchor member within hole H of bone B may be employed. Cleated members 42 of FIG. 2A, which are depicted in most of the embodiments herein, are merely one exemplary means for securing. Other alternative means for securing may be used in conjunction with the apparatus and methods of the present invention. As an example, the bone anchor member may employ one or more radially expandable members that extend into the surrounding bone.

Further, while some of the embodiments of the present invention describe use of a bone anchor member only, and other embodiments describe use of a bone anchor member and a plug portion, many of these features may be interchanged. It will be apparent to one skilled in the art that many embodiments depicting a bone anchor member only may be performed using a bone anchor member and plug portion, and vice versa.

Also, for those embodiments described hereinabove having a bone anchor member and a plug portion, it will be apparent to those skilled in the art that the suture ends may be tensioned either before or after the plug portion is inserted into the bore of the bone anchor member.

It will also be apparent to one skilled in the art that the plug portion may be securely disposed within the bore of the bone anchor member using various means not specifically disclosed herein. For example, after the plug portion is inserted into the bore of the bone anchor member, an adhesive, for example, cyanoacrylate, epoxy, bone cement and so forth, may be delivered to affix the plug portion to the bone anchor member. Alternatively, an exterior surface of the plug portion may be coated with a biocompatible adhesive that affixes to the bone anchor member after the plug portion is inserted into the bore of the bone anchor member. In yet a further alternative embodiment, heat energy may be applied to fuse the plug portion to the bone anchor member. It will be apparent to one skilled in the art that still further means for securing the plug portion to the bone anchor member may be employed.

In still further embodiments of the present invention, the objective of the present invention may be achieved using multiple bone anchor members, or multiple bone anchor members coupled to respective plug portions. In each embodiment, one or more sutures may be coupled between a desired tissue region and the bone anchor member or plug portion. If multiple sutures and bone anchor members are employed, enhanced sequential tensioning of the tissue may be achieved.

Finally, while the above-described embodiments reference use of apparatus and methods for facilitating attachment of tissue to bone, it will be apparent to one skilled in the art that such apparatus and methods may also be used to secure tissue to tissue and bone to bone.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

1. A method of securing suture to bone, comprising the steps of:
providing a bone anchor and a length of suture, the anchor having a proximal end, a distal end and a first suture passage;
positioning a first suture portion of the length of suture in the first suture passage, the length of suture having a first side and a second side, the first and second sides extending from each side of the first suture;
coupling the first side of the length of suture to a tissue structure;
introducing the bone anchor into a bone, the distal end being embedded in the bone and the proximal end being exposed, the first and second sides extending from the proximal end of the bone anchor;
pulling the second side thereby increasing tension in the first side of the length of suture; and
releasing tension on the first side of the length of suture, the first suture portion being automatically locked in the first passage upon releasing tension on the first suture.

2. The method of claim 1, wherein:
the pulling step is carried out with the first and second sides of the length of suture extending from the proximal end of the bone anchor, at least the first side of the length of suture being free to extend in any direction from the proximal end of the bone anchor.

3. The method of claim 1, wherein:
the providing step is carried out with the bone anchor having a second suture passage.

4. The method of claim 3, further comprising the steps of:
positioning a second suture portion of a second length of suture in the second suture passage, the second length of suture also having a first side and a second side extending from the proximal end of the bone anchor;
pulling the second side of the second length of suture to increase tension in the first side of the second length of suture;
wherein the pulling steps are carried out independently to independently tension the first and second lengths of suture.

5. The method of claim 4, wherein:
the positioning steps are carried out with the first and second lengths of suture portions being part of the same continuous piece of suture.

6. The method of claim 1, wherein:
the providing step is carried out with the first suture passage permitting the first suture portion to be advanced in one direction and preventing advancement in the other direction.

7. The method of claim 1, wherein:
the providing step is carried out with the bone anchor having a first part and a second part; and
the positioning step is carried out by moving the first and second parts away from each other thereby permitting positioning of the first suture portion in the first suture passage.

8. A method of securing suture to bone, comprising the steps of:
providing a bone anchor having a first suture passage and a suture locking mechanism movable from an unlocked position to a locked position;
positioning a first suture portion of the length of suture in the first suture passage, the length of suture having a first side and a second side extending from each side of the first suture;
coupling the first side of the length of suture to a tissue structure;
introducing the bone anchor into a bone, the distal end being embedded in the bone and the proximal end being exposed, the first and second sides extending from the proximal end of the bone anchor;
adjusting the tension in the first side of the length of suture after the introducing step by manipulating the second side; and
locking the first suture suture portion in the first suture passage with the suture locking mechanism moved to the locked position after the adjusting step.

9. The method of claim 8, wherein:
the adjusting step is carried out with the first and second sides of the length of suture extending from the proximal end of the bone anchor, at least the second side of the length of suture being free to extend in any direction from the proximal end of the bone anchor.

10. The method of claim 8, wherein:
the providing step is carried out with the suture locking mechanism being naturally biased toward the locked position.

11. The method of claim 8, further comprising the step of:
repeating the adjusting and locking steps.

12. The method of claim 8, wherein:
the providing step is carried out with the bone anchor having a first part and a second part, the first part having a recess in which the second part is positioned, the first part also having a longitudinal axis, the second part being movable longitudinally relative to the first part when moving between the locked and unlocked positions.

13. The method of claim 12, wherein:
the providing step is carried out with the second part moving toward the distal end when moving the suture locking mechanism to the unlocked position.

14. The method of claim 8, wherein:
the providing step is carried out with the bone anchor having a first part and a second part, the first part having a recess in which the second part is positioned, the first part also having a longitudinal axis, the second part being rotatable about the longitudinal axis relative to the first part when moving between the locked and unlocked positions.

15. The method of claim 14, wherein:
the providing step is carried out with the second part having a deflectable portion, the deflectable portion being deflected distally to move the suture locking mechanism to the locked position.
